(19) United States
(12) Patent Application Publication
(10) Pub. No.: US 2008/0305168 A1
(43) Pub. Date: Dec. 11, 2008

(54) In-Situ Melting and Gelling Tablet Composition For Oral Care

(76) Inventors: Kyo-Tae Moon, Chungcheongnam-do (KR); Taek-Kyun Kang, Daejeon (KR); Jae-Hyun Ahn, Daejeon (KR); Sang-Jin Kang, Seoul (KR); Sang-Nyun Kim, Daejeon (KR); Sug-Youn Chang, Daejeon (KR); Moon-Jung Rang, Daejeon (KR)

Correspondence Address: BIRCH STEWART KOLASCH & BIRCH, PO BOX 747, FALLS CHURCH, VA, 22040-0747, US

(21) Appl. No.: 12/161,384

(22) PCT Filed: Feb. 8, 2007
(86) PCT No.: PCT/KR07/00687

§ 371 Date: Jul. 18, 2008

(30) Foreign Application Priority Data
Feb. 10, 2006 (KR) 10-2006-0013113
Feb. 10, 2006 (KR) 10-2006-0013122
Nov. 1, 2006 (KR) 10-2006-0107099

(51) Int. Cl. A61K 9/20; A61P 1/02

(52) U.S. Cl. 424/465

(57) ABSTRACT

Disclosed herein is an oral care composition that provides pleasant cooling sensation and easy delivery of an oral care active ingredient. The tablet-type oral care composition of the present invention is prepared by compressing porous plastic granules at a pressure of 500 kg/cm2 or less and undergoes in situ melting and gelling by saliva or water in the buccal cavity, or by chewing. Upon direct administration of the composition to the buccal cavity, the composition undergoes melting and gelling by water or saliva, or by chewing action, and works to maintain oral health via gargling or mouth washing. Therefore, the formulation of the present invention advantageously reduces inconvenience of conventional liquid or ointment-like oral care products and also provides easy portability.

### TECHNICAL FIELD

The present invention relates to an oral care composition that provides pleasant cooling sensation and easy delivery of an oral care active ingredient. More specifically, the present invention relates to an oral care composition that undergoes in-situ melting and gelling by water or saliva, or the chewing action to thereby allow mouth washing or gargling.

### BACKGROUND ART

Generally, oral care agents may be broadly classified into four types of toothpaste, mouth wash, film and spray. These oral care products are usually applied for esthetic purposes including teeth whitening, prevention of plaque and calculus deposition and inhibition of halitosis and for therapeutic or prophylactic purposes of dental caries and periodontal diseases.

Meanwhile, fast-melting tablets have been produced in the pharmaceutical industry for convenient internal use of tablet preparations including pharmaceutical preparations and health products. Toshihiro Shimizu et al. (Chem. Pharm. Bull. 51(10) (2003)) pointed out that fast-disintegrating tablets are greatly beneficial for patients who have difficulty in swallowing conventional tablet dosage forms, with statement that preparation of such fast-disintegrating tablets involves manufacturing processes such as tablet molding, freeze-drying, spray-drying, disintegrant addition, sublimation, use of sugar-based excipients, and the like.

Production of fast-melting tablets may employ manufacturing technologies such as freeze-drying, molding, compression process, and the like.

First, freeze-drying (lyophilization) is a process removing solvents from frozen drug solutions or frozen drug suspensions containing excipients. The tablets obtained by freeze-drying are usually very light and have porous and plastic structures that allow rapid dissolution. However, freeze-drying is a relatively expensive process, and the final tablet dosage forms suffer from high fragility and low mechanical strength, thus making it difficult to achieve blister packaging.

In the molding process, materials are dissolved in water or ethanol and the wet mass is compressed at a pressure lower than conventional tablet compression pressure, followed by drying to obtain a fast-melting tablet. Major components of the molded tablet are typically water-soluble. The molded tablet is porous and therefore such a porous structure allows easy penetration of water and promotes dissolution of the tablet. Whereas, as disclosed in U.S. Pat. No. 5,082,667 issued to Van Scoik et al., the molded tablet suffers from various problems associated with handling, transportation and distribution due to very low mechanical strength. Further, manufacture of the molded tablet involves more complicated processes than conventional compression processes.

Manufacture of the fast-melting tablets by means of a conventional tablet press is a very attractive method in terms of production costs and technology transfer. In a conventional tablet compression process, the porosity of the tablet is not an important factor and high-pressure compression is applied to increase strength of the tablet. Therefore, it was impossible to obtain the fast-melting tablet. As examples of methods to achieve high porosity, there are granulation, utilization of certain soluble materials, and post-treatment following manufacture of the final tablet. Kinds and characteristics of these methods will be briefly illustrated hereinafter.

As granulation methods, fluidized-bed wet granulation disclosed in U.S. Pat. No. 6,149,938 issued to Bonadeo et al., dry granulation disclosed in U.S. Pat. No. 5,939,091 issued to Eoga et al., spray-drying granulation disclosed in U.S. Pat. No. 6,207,199 issued to Allen et al., and flash heat techniques disclosed in U.S. Pat. No. 6,048,541 assigned to Fuisz Technologies Ltd. (Chantilly, Va.) are employed in preparation of fast-melting tablets.

In the method using specific excipients, it is important to select water-insoluble calcium salts, specific combination of disintegrants and specific sugar-based excipients. U.S. Pat. No. 6,596,311 issued to Dobetti et al. discloses utilization of water-insoluble inorganic excipients as the main component for fast disintegrating tablets. Regarding a method via adjustment of sugar or sugar derivatives, Chang, R. -K. et al. (Pharmaceutical Development & Technology, 24: 52-58, 2000) reported that nearly all formulations for quick-dissolving tablets use sugar or sugar derivatives. Next, in the method using disintegrants, nearly all formulations for rapidly dissolving tablets incorporate an effervescent acid-base couple or combinations of several non-effervescent disintegrants. Examples of such non-effervescent disintegrants may include carboxymethylcellulose, cross-linked polyvinylpyrrolidone, starch, modified starch, carboxymethyl starch, microcrystalline cellulose, and the like.

Compaction and subsequent treatment are carried out to strengthen brittle tablets by various after-treatments such as sublimation, sintering and humidity treatment after preparation of tablets at a low compression pressure. U.S. Pat. No. 5,762,961 issued to Roser et al. discloses a method for producing rapidly soluble tablets using volatile materials. The humidity control process is carried out taking advantage of the fact that sugars undergo phase transition from an amorphous state to a crystalline state when their solution is spray-dried or used as a binder solution. That is, control of humidity during drying and granulation processes leads to a change of sugar from the amorphous state to the crystalline state, which consequently increases the tablet strength substantially. U.S. Pat. No. 6,465,010 issued to Lagoviyer et al describes a process that increases the tablet strength by sintering the tablet components at high temperatures and resolidifying after the temperature decreases subsequently.

Yet little is known about research related to application of the aforesaid fast-melting tablet manufacturing technologies to oral care agents. For conventional oral care agents, an attempt was made to prepare a fast-melting tablet using effervescent couples and disintegrants. In this case, the conjugate acid used for effervescence has unpleasant effects on common feelings of consumers and therefore there is an urgent need for improvements in feeling of use and friendliness. Specifically, US Patent Applications 2004/0101493 A1 and 2004/0101494 A1 of Douglas C. S. et al. disclose chewable solid unit dosage forms and methods for delivery of active agents into occlusal surfaces of teeth. According to these patents, the oral care active agent is delivered directly to, and retained on, the occlusal surfaces of teeth including teeth interstices and molar teeth, which may conventionally occur after chewing by the subject. However, these preparations may give rise to foreign-body sensation after use thereof thereby leading to unpleasant effects to consumers. In addition, these preparations are not fast-melting tablets and thus suffer from a disadvantage in that they cannot exhibit gel-like properties, as shown in conventional toothpaste, within several seconds. Therefore, there is a need for improvements in medication feeling and convenience of the consumers.

### DISCLOSURE OF INVENTION

### Technical Problem

Therefore, the present invention has been made in view of the above problems, and it is an object of the present invention to provide an oral care composition in the form of an in-situ melting and gelling tablet which is distinct from a conventional fast-melting tablet; that is, is superior in patient medication acceptability and convenience and can be used for various applications of tablets including chewable tablets, oral melting and gelling tablets, toothbrush-attached melting and gelling tablets, and the like.

### Technical Solution

In accordance with a first aspect of the present invention, the above and other objects can be accomplished by a tablet-type oral care composition which is prepared by compressing porous plastic granules consisting essentially of a material for porous plastic granules, a binder, a gelling agent and a water penetration enhancer under a pressure of 500 kg/cm² or less and undergoes in-situ melting and gelling by water or saliva in oral cavity.

In accordance with a second aspect of the present invention, there is provided a tablet-type oral care composition which is prepared by compressing porous plastic granules consisting essentially of a material for porous plastic granules, a binder, a gelling agent, a water penetration enhancer and an anti-adhesive agent to teeth under a pressure of 500 kg/cm² or less and undergoes in-situ melting and gelling by chewing in the presence of water or saliva in oral cavity.

In accordance with a third aspect of the present invention, there is provided a tablet-type oral care composition which is prepared by compressing porous plastic granules consisting essentially of a material for porous plastic granules, a binder, a gelling agent, a water penetration enhancer, an anti-adhesive agent to teeth and a humectant under a pressure of 500 kg/cm² or less and undergoes in-situ melting and gelling by water or saliva in oral cavity, or by chewing.

Toothpaste is a representative oral care agent and realizes dental health by spreading the paste on a toothbrush and brushing teeth. The present invention provides a tablet-type oral care agent that brings about pleasant refreshing effects and alleviates or prevents oral diseases even with simple water gargling as well as teeth brushing, due to in-situ melting and gelling of the tablet by the chewing action in the oral cavity or by water or saliva on the toothbrush or in the oral cavity.

The tablet-type oral care agent according to the present invention provides all effects including oral cleaning, prevention of dental caries and prophylaxis of periodontal diseases that are usually possessed by conventional oral care agents. In addition, the tablet-type oral care agent of the present invention is a novel conceptual oral care product distinct from the conventional oral care products, in that in-situ melting and gelling of the tablet is achieved by water or saliva or melting and gelling of the tablet is facilitated by the chewing action.

In order to meet physical properties necessary for in-situ melting and gelling tablet, high porosity for easy penetration of water should be secured such that water penetrates into the core of the tablet within several seconds and reasonable strength should be secured to provide easy handling. The fast-melting tablet of the present invention may comprise a high-porosity plastic granular material, a water/saliva penetration enhancer, a binder, a gelling agent, an anti-adhesive agent, a humectant, an abrasive, a forming agent, a fragrance, a sweetening agent and an active ingredient.

Generally, the fast-melting tablet used in the pharmaceutical industry is designed to secure rapid absorption of a drug within the oral cavity and therefore melting/gelling as shown in the toothpaste is not a primary concern. In the present invention, the tablet composition is contrived to have a gel texture similar to that of conventional toothpaste.

It can be said that both of melting and gelling are competitive processes requiring water or saliva. Therefore, a part of water or the saliva should be used for dissolution, and the remainder should be used for gelling. However, it is technically difficult to achieve simultaneous melting and gelling.

In order to obtain in-situ melting and gelling properties in the present invention, an attempt was made to produce a high-strength tablet by preparation of porous plastic granules via granulation, inducement of rapid gelling via the use of a highly hydrophilic polymer, use of a water penetration enhancer, and preparation of a high-strength tablet by low-pressure tablet compression processes using a binder solution during a manufacturing process of tablet so as to ensure melting and gelling thereof. Further, in order to secure basic functions of the conventional toothpaste, the tablet composition of the present invention is made to contain an abrasive, a forming agent and a fragrance to thereby impart superior feeling of use to consumers.

The tablet-type oral care agent of the present invention takes advantage of the fast-melting tablet technology and a fast-gelling technology. The technology for production of the fast-melting tablet includes preparation of the porous plastic granules, and preparation of a high-strength tablet at low compression pressure using a water penetration enhancer and a binder solution.

The preparation of the porous plastic granules employs materials that are readily soluble or dispersible upon contact with water or saliva. The materials to be used should be harmless as a drug or food material. When the plastic granules are compressed into tablets using a tableting machine, plastic deformation of the granules dramatically increases by particle-particle contact.

When it is desired to use a polymer as the porous plastic material in the composition of the present invention, it must ensure that water penetration into the core of the tablet is not blocked by water film formation due to a viscosity increase which may occur on the surface of the tablet during dissolution of the tablet in water. Preparation of such a tablet involves use of a water/saliva penetration enhancer in a given ratio. The water penetration enhancer serves to prevent inhibition of penetration of viscous water on the surface of the tablet. Generally, the water/saliva penetration enhancer and the porous plastic granules are made of different materials, but in some cases they can be the same materials.

Even though use of the porous plastic granules and the water/saliva penetration enhancer may bring about plastic deformation, thereby forming a tablet via binding between raw materials, it is very important to use a binder so as to elicit strong bond between the raw materials. The binder serves to safely maintain a porous structure of the porous plastic granules and functions of the water/saliva penetration enhancer. No use of the binder results in segregation between the aforementioned materials. A conventional binder is in the liquid or ointment-like semi-solid state. Therefore, it is very important to maintain inherent properties of the above two materials by the binder. In order to achieve this purpose, it is possible to use a high concentration of the binder showing substantially no reactivity with water. Alternatively, by using a relatively low concentration of the binder and a short mixing time, it is also possible to prevent loss of inherent properties of the material for porous plastic granules and the water/saliva penetration enhancer which may occur due to use of the binder.

During preparation and extrusion of the porous plastic granules, it is possible to bring about changes in physical properties of the resulting granules by varying an introduction point of each component. In addition, the introduction position of the components may vary depending upon desired physical properties of the granules.

Hereinafter, individual components an in-situ melting and gelling oral care agent in the form of a fast-melting tablet according to the present invention will be described in more detail.

Materials for Porous Plastic Granules

High-porous plastic granules added to a composition of the present invention are prepared not so as to undergo deformation even with application of external pressure of 500 kg/cm².

The porous plastic materials should have porosity of 0.14 or higher and a density of 0.86 or less. In addition, the plastic material should undergo plastic deformation while retaining its shape and size when it is compressed using a mold having a diameter of 1.27 cm at a pressure of 500 kg/cm² or less. Generally, upon compression into a tablet under the pressure of more than 675 kg/cm², this leads to destruction of pores, thereby making it impossible to maintain properties of the fast-melting tablet.

Preferably, the porous plastic material is water-soluble. A content of the porous plastic material with high water-solubility is preferably in a range of 1 to 98% by weight, and more preferably 20 to 95% by weight, based on the total weight of the tablet. If a content of the porous plastic material is lower than 1% by weight, it cannot provide enough contacts with other components, thereby resulting in excessively low strength of the resulting tablet, Whereas, if a content of the porous plastic material is higher than 98% by weight, then additional components, such as a water penetration enhancer, a binder, an active ingredient and other additives, cannot be included.

The porous plastic material used in the composition of the invention is commercially available or can be easily made by various methods, e.g., spray-drying, fluidized-bed granulation, and so forth. Examples of the soluble plastic material that can be used in the present invention may include, but are not limited to, saccharides such as fructose, lactitol, maltitol, maltose, mannitol, sorbitol, sucrose, erythritol and xylitol, as well as organic polymers such as maltodextrin, dextrin, ethylcellulose, polymethylmethacrylate and pregelatinized starch (e.g., LYCATAB by Roquette America, Inc.). Among these materials, sorbitol is a saccharide having the highest dissolution rate, whereas erythritol is ideal for water/saliva penetration even though its solubility is not high.

Other materials that can form suitable porous plastic structures include gum arabic, xanthan gum and its derivatives, guar gum and its derivatives, seaweed gum, carrageenan, dextran, gelatin, alginate, pectin, starch and starch derivatives (e.g., hydroxypropyl starch and hydroxyethyl starch), cellulose esters (e.g., carboxymethyl cellulose or cellulose ether hydroxyethyl-methyl celluloses), homo- or co-polymers of an unsaturated acid (e.g., acrylic acid or a salt thereof), homo- or co-polymers of an unsaturated amide (e.g., acrylamide), homo- or co-polymers of acrylic imine, a vinyl polymer (e.g., polyvinyl alcohol), homo- or co-polymers of a vinyl ester (e.g., vinyl pyrrolidone, vinyl oxazolidone, vinyl methyl oxazolidone, vinyl amine and vinyl pyridine), alkyl glycol and polyalkylene oxide (e.g., polyethylene oxide) and oxyethylene alkyl ester, dextrate, dextrin, dextrose, microcrystalline cellulose, silicified microcrystalline cellulose, powdered cellulose, cellulose acetate, calcium sulfate, calcium carbonate, dibasic calcium phosphate, tribasic calcium phosphate, a calcium salt of carboxymethyl cellulose and silica. Inorganic materials, such as calcium carbonate and silica, exhibit rapid absorption of water but show a weak binding force therebetween.

Water/Saliva Penetration Enhancers

A water/saliva penetration enhancer in the composition of the present invention is employed to bring about fast disintegration of a tablet. The water/saliva penetration enhancer is evaluated as follows: a 200 mg of a candidate material is compressed at 135 kg in a 1.27-cm diameter mold, and several water drops are fallen on a surface of the resulting tablet. When the water drop does not spread on the surface of the tablet, the candidate material cannot be used as the water/saliva penetration enhancer. When water spreads or is absorbed on the surface of the tablet within 60 seconds, the candidate material can be used as the water/saliva penetration enhancer.

For use in production of fast-melting tablets, the water penetration enhancer should be highly water-soluble, or otherwise it should at least be highly dispersible via rapid absorption of water. Generally, the water/saliva penetration enhancers are highly water-soluble carbohydrates, which are often used as excipients. Without particular limitation, any type of carbohydrates may be used in the composition of the present invention. However, for oral care-related formulations, it is common to use a minimum amount of a component that produces an organic acid as a by-product through the metabolism of Streptococcus mutans, thereby being capable of causing dental caries. Examples of such carbohydrates may include dextrate, dextrin, dextrose, fructose, lactitol, lactose, maltitol, mannitol, sorbitol, sucrose, erythritol, and xylitol. Examples of materials that are poorly water-soluble, but have high water-dispersibility and high water-transfer capacity, may include microcrystalline cellulose, silicified microcrystalline cellulose, powdered cellulose, cellulose acetate, calcium sulfate, calcium carbonate, silica, dibasic calcium phosphate, tribasic calcium phosphate, a calcium salt of carboxymethyl cellulose, and cross-linked polyvinyl pyrrolidone. Various combinations of carbohydrates and polymers may also be used. Preferred materials for the water/saliva penetration enhancer are porous materials that can be directly compressed into tablets. In addition, materials capable of transferring water such as inorganic materials including calcium carbonate, silica and dibasic calcium phosphate and organic materials including cross-linked polyvinyl pyrrolidone, microcrystalline cellulose, cellulose and erythritol are also excellent water/saliva penetration enhancers.

In the tablet composition of the present invention, the action of the water penetration enhancer in conjunction with the mechanical tablet destruction operation such as chewing action is effected in the oral cavity. Therefore, the composition of the present invention may exhibit no difference in the melting and gelling time period even when the content of the water penetration enhancer is low as compared to conventional tablets where in-situ melting and gelling are achieved only by water or the saliva. A content of the water penetration enhancer is preferably in a range of 1 to 98% by weight, and more preferably 20 to 80% by weight, based on the total weight of the tablet. If a content of the water penetration enhancer is lower than 1% by weight, it cannot provide water penetration into the core of the tablet. On the other hand, if a content of the water penetration enhancer is higher than 98% by weight, then components other than the water penetration enhancer cannot be included in the tablet composition and undesirably excessive amounts of saliva or water greater than production of saliva in the oral cavity are necessary for fast melting of the tablet.

In the composition of the present invention, where chewing is necessarily required in in-situ melting and gelling as compared to when chewing is not necessary, it is possible to enhance the preference and use feeling of consumers by using a small amount of a water-insoluble water penetration enhancer and increasing an amount of a water-soluble water penetration enhancer. Among the above-mentioned water penetration enhancers, the water-insoluble water penetration enhancer may be preferably used in an amount of 0.01 to 7% by weight.

Whereas, when the chewing action is not essentially required in the in-situ melting and gelling, it is necessary to employ a relatively large amount of the water-insoluble water penetration enhancer.

Binders

The binder of the present invention is one widely used in conventional tablet compression processes. In the composition of the present invention, the primary function of the binder is to enhance binding force between all raw materials including high-porous plastic granules and water/saliva penetration enhancer, thereby preventing separation of individual components from one another, and to obtain a high-strength in-situ melting and gelling tablet even at low compression pressure. A content of the binder is in a range of 1 to 90% by weight, based on the total weight of the tablet.

The binder may be in the liquid or semi-solid form, depending on granulation methods to be employed. The most important one of requirements necessary for the binder is to cause minimum destruction of the pore structure of the thus-obtained porous plastic material. This purpose can be achieved, for example, by simply lowering the water activity using a high concentration of the binder that was saturated to a level above the solubility of water or by uniformly dispersing a solution of the binder at a low concentration for a short period of time. A simple test can be performed to examine damage to the porosity and solubility. 1 mL of the binder solution is added to 0.5 g of the porous plastic material; if the porous material is not completely dissolved within 10 seconds while maintaining the porous structure intact, this binder solution can be used in the composition of the present invention.

After the wet granules are dried, the solidified binder preferably dissolves quickly upon contact with water. The type and amount of the binder for wet granulation may be adjusted depending upon desired physical properties, such as high plasticity and good binding properties. Other pharmaceutically acceptable organic solvents, such as ethyl alcohol, may also be used as a solvent for the binder solution, which may lead to less destruction of the pore structure of the porous materials. Examples of binder materials may include carbohydrates listed in the water penetration enhancer component, and polymers such as acacia, alginic acid, carbomer (Carbopol), carboxymethyl cellulose, cellulose, dextrin, ethyl cellulose, gelatin, hydroxyethyl cellulose, hydroxypropyl cellulose, hydroxypropyl methyl cellulose, methyl cellulose, polydextrose, polyethylene oxide, polyvinyl pyrrolidone and sodium alginate.

Gelling Agents

A gelling agent that can be used in the present invention may be water-soluble polymers or natural polymers, such as gum, that are easily swellable in water/saliva and then undergo gelling. The gelling agent in the composition of the present invention greatly affects the gel texture that is most important for physical properties of the tablet in conjunction with fast-melting properties of the tablet, and is the most important factor that determines feeling of use in final products. A requirement necessary for the gelling agent is to have the gel texture within several tens of seconds by the action of water/saliva and the following method can be employed to confirm this: 1 mL of water drops on a tablet of the gelling agent weighing 200 mg, If the surface of the tablet exhibits gel-like properties within 10 seconds, such a material can be used as the gelling agent for the present invention. Another important factor of the gelling agent is to ensure that penetration of water or saliva is not blocked due to formation of a hydrated membrane on the surface of the tablet when the gelling agent reacts with water or saliva to turn into a gel.

In order to achieve rapid gelling, the gelling agent should be hydrophilic and should be gelated by absorbing a small amount of water. In order to prevent that the gelling agent blocks the pores of the porous plastic granules during the wet granulation process, the wet granulation should be completed within 5 min. Examples of the gelling agent that can be used in the present invention may include acacia gum, agar gum, gellan gum, guar gum, pectin, gelatin, alginic acid, sodium alginate, carboxymethyl cellulose, a polyvinyl maleic acid/maleic anhydride copolymer, Carbopol, polyethylene glycol, polyvinyl pyrrolidone, polyethylene oxide, xanthan gum, carrageenan, super porous hydrogel, and the like. These materials may be used alone or in any combination thereof. Among these materials, xanthan gum (CP Kelco, USA) and Carbopol (Noveon, USA), carboxymethyl cellulose (Hercules, USA) are commercially available.

The gelling agent is preferably used in an amount of 0.1 to 10% by weight, based on the total weight of the tablet. Where a content of the gelling agent is lower than 0.1% by weight, a sufficient viscosity is not secured and it is difficult to maintain the shape and size of the tablet after dissolution thereof. On the other hand, where a content of the gelling agent is higher than 10% by weight, it is difficult to achieve sufficient penetration of water into a core of the tablet, thereby resulting in gelation only on the surface of the tablet, simultaneously with undesirable sticking of the binder to the teeth upon chewing the tablet.

Anti-Adhesive Agents to Teeth

In connection with a tablet-type oral care composition of the present invention, when chewing action is required in in-situ melting and gelling of the tablet, the oral care product may remain in interstices between teeth, which consequently leads to deterioration in feeling of use and perceived quality after use by consumers. In order to cope with these disadvantages, an anti-adhesive agent is added to minimize sticking of materials to teeth which occurs upon chewing the tablet.

Examples of components for the anti-adhesive agent may include surfactants such as glyceryl monooleate, glyceryl monostearate, and the like, which can be used to prevent sticking of tablet components to the surface of teeth that is hydrophilic.

The anti-adhesive agent is used in a range of 0.01 to 10% by weight, based on the total weight of the tablet. Where a content of the anti-adhesive agent is lower than 0.01% by weight, it is difficult to exert sufficient anti-adhesive properties of the agent. On the other hand, where a content of the anti-adhesive agent is higher than 10% by weight, this leads to adverse effects on taste and use feeling of the tablet.

Humectants

Due to use of the porous plastic granules, the tablet-type oral care composition of the present invention may give rise to powdery feeling caused by abrasives after drying of the product. In order to solve this problem, a humectant may be used which is a component capable of maintaining moisture content of the granule at a constant level. Examples of materials that can be used for the humectant may include glycerin, polyethylene glycol, propylene glycol, sorbitol, and the like.

The humectant may be used in an amount of 0.01 to 20% by weight, based on the total weight of the tablet. Where a content of the humectant is lower than 0.01% by weight, it is difficult to exert sufficient anti-adhesive properties of the humectant. On the other hand, where a content of the humectant is higher than 20% by weight, this may result in overwetting of the porous plastic granules.

Lubricants

A lubricant is added to prevent foreign materials from being caught in a punch of a tableting machine during a compression process. As examples of the lubricants, sodium lauryl sulfate, magnesium stearate, stearic acid, and the like may be used. Typically, the lubricant may be used in an amount of 0.1 to 5% by weight, based on the total weight of the tablet.

Forming Agents/Surfactants

A forming agent and a surfactant in the oral care agent serve to not only clean teeth, but also take part in formation of bubbles that is one of the most important attribute in the oral care agent. Generally, the forming agent includes an anionic surfactant, a cationic surfactant, a non-ionic surfactant, an amphoteric surfactant, and a zwitterionic surfactant. The forming agent may be a single material or a combination of two or more materials. A content of the forming agent in the composition of the present invention is in a range of about 0.001 to 20% by weight, preferably 0.1 to 5% by weight, based on the total weight of the tablet.

As the most representative examples of the forming agent that may be optionally used in a safe and effective amount, mention may be made of the anionic surfactant such as sodium lauryl sulfate and sodium coconut monoglyceride sulfonate. As other suitable examples of the anionic surfactant, mention may be made of sarcosinate, e.g. sodium lauroyl sarcosinate, taurate, sodium lauryl sulfoacetate, sodium lauroyl isethionate, sodium laureth carboxylate and sodium dodecyl benzene sulfonate. In addition, cocamidopropyl betaine, Poloxamer, sorbitan monooleate, PEG-40 sorbitan isostearate or a mixture thereof may also be used as the surfactant.

Abrasives

An abrasive is very important for cleaning action of oral care products including removal of plaques, food debris, and the like. The abrasive should not damage the enamel of teeth and have compatibility with other components of the composition. The abrasive used in the composition of the present invention is selected from materials that do not cause excessive erosion of tooth dentin leading to tooth sensitivity and is used in a suitable amount. Currently, examples of materials for the abrasive suitable for use in oral care formulations may include silica, calcium pyrophosphate, aluminum hydroxide, tribasic calcium phosphate, dibasic calcium phosphate dihydrate and anhydride, and calcium carbonate, which are in the form of gels or precipitates. In addition to those materials, natural fibrous materials such as cellulose and derivatives thereof, and natural materials such as shell powders of eggs and shellfish may also be used as the abrasive. In the present invention, a content of the abrasive in the oral care composition is in a range of about 5 to 70% by weight, preferably 15 to 50% by weight, based on the total weight of the composition.

Flavoring Agents and Sweetening Agents

The composition of the present invention may be formulated with addition of flavoring agents and sweetening agents so as to meet the preference and palatability of consumers. As the flavoring agent, there may be used mints including peppermint and spearmint, Wintergreen, Anis, menthol, thymol, methyl salicylate, eucalyptol, eugenol, polypropyleneglycol, melon, strawberry, orange, vanillin, and the like. Typically, the flavoring agent may be used in a range of 0.001 to 10% by weight, based on the total weight of the composition.

Further, the sweetening agent may be added to the composition of the present invention in order to achieve pleasing mouth-feel and good taste masking. Examples of the sweetening agents may include saccharin, sucralose, sucrose, xylitol, sorbitol, lactose, mannitol, maltitol, erythritol, aspartame, taurine, saccharin salts, D-tryptophan, and the like. These materials may be used alone or in any combination thereof. Among the saccharin salts, saccharin sodium is most widely used as the sweetening agent. Typically, the sweetening agent may be used in a range of 0.001 to 20% by weight, based on the total weight of the tablet composition.

Active Ingredients

As an active ingredient, also active pharmaceutical ingredient (or API), there may be employed various substances effective for anti-caries, prevention of gingival and periodontal diseases, prevention of calculus deposition, whitening of teeth, and the like, depending upon desired applications of oral care formulations.

The active ingredients for prevention of dental caries may include compounds that have received U.S. FDA (Food and Drug Administration) approval as safe and effective materials, including fluoride-containing compounds. As examples of compounds that can be used as a source of fluoride ions, mention may be made of sodium fluoride, sodium monofluorophosphate, stannous fluoride, and amine fluoride. Even though there may be some differences between individual countries, a single fluoride source or a combination of two or more sources is typically used to provide a fluoride ion concentration of preferably 850 to 1500 ppm.

A re-calcifying agent may also serve as an anti-caries agent. Recalcification is a regeneration and recovery process of hydroxyapatite that is a major component of teeth. Hydroxyapatite is largely composed of divalent calcium cations (Ca²⁺) and divalent phosphate anions (PO₄⁻²). Therefore, any material may be used as the re-calcifying agent, so long as it contains either or both of calcium divalent ions and phosphate anions, such that calcium ions and phosphate ions can be simultaneously supplied or the chemical equilibrium in the oral cavity can be shifted toward production of hydroxyapatite. Examples of materials that can provide calcium ions and phosphate ions may include hydroxyapatite, dicalcium phosphate, calcium chloride, casein phosphopeptide, calcium glycerophosphate, monobasic sodium phosphate, dibasic sodium phosphate, tribasic sodium phosphate, monobasic potassium phosphate, dibasic potassium phosphate, tribasic potassium phosphate, and the like. Typically, the re-calcifying agent is preferably used in an amount of 0.001 to 20% by weight, based on the total weight of the composition. If a content of the re-calcifying agent is lower than 0.001% by weight, it is difficult to achieve sufficient re-calcifying effects. If a content of the re-calcifying agent is higher than 20% by weight, this may result in loss of inherent properties of the tablet.

One of desired applications of the oral care products is to not only alleviate ongoing gingival and periodontal diseases but also prevent the onset of such diseases by sterilizing or anti-inflammatory action against harmful bacteria inhabiting the oral cavity. For this purpose, thymol, cyclohexidine, cetylpyridinium chloride, triclosan, xanthorrhizol, and the like may be used which are known as antibacterial agents. For anti-inflammatory action, vitamins and enzymes may also be used.

Materials exhibiting whitening effect in addition to therapeutic/prophylactic effects on dental diseases, for example hydrogen peroxide, carbamide peroxide, calcium peroxide or the like may be employed. In order to obtain inhibitory effects on calculus deposition, sodium pyrophosphate, acidic sodium pyrophosphate, potassium pyrophosphate, sodium metaphosphate, or the like may also be used. Typically, these active ingredients are used in an amount of 0.001 to 10% by weight, based on the total weight of the composition.

The fast-melting tablet-type in-situ melting and gelling oral care agent according to the present invention is prepared using the aforementioned components. FIGS. 1 to 4 schematically show a process for preparing the tablet. FIG. 1 shows a general production process of the in-situ melting and gelling tablet of the present invention, FIG. 2 shows co-introduction of a gelling agent with a binder solution, FIG. 3 shows introduction of a gelling agent after preparation of porous plastic granules, and FIG. 4 shows production of the tablet after preparation of two types of granules.

Hereinafter, production of the oral care formulation of the present invention will be illustrated with reference to the process of FIG. 1.

First, necessary amounts of solid raw materials for use in preparation of porous plastic granules are precisely weighed and mixed in pharmaceutical-grade machinery, a high-speed mixer while adding a binder solution. Mixing is continued until all powder materials are bound without causing damage to the porosity of particles. The mixed materials are screened through a sieve having a size of 10 to 50 mesh and dried to have a moisture content of less than 10% at room temperature or in a conveyer belt. Dried granules are screened again through a sieve having a size of 10 to 50 mesh to obtain porous plastic granules. The thus-obtained plastic granules are mixed with a flavoring agent, an active ingredient and a glidant, and the resulting mixture is compressed at a relatively low pressure of 500 kg/cm² or less, thereby obtaining an in-situ melting and gelling oral care tablet containing porous plastic granules. The compression pressure of 500 kg/cm² is a maximum pressure to obtain granules having no problem associated with handling, transportation and distribution while maintaining the porosity of porous plastic granules.

FIGS. 2 and 3 show alteration in an introduction step of the gelling agent in the process of FIG. 1. The process of FIG. 4 produces the in-situ melting and gelling tablet by preparing two types of granules and compressing the granules at low pressure into a tablet.

The thus-prepared tablet-type oral care formulation according to the present invention undergoes in-situ melting and gelling within 30 seconds by saliva in the oral cavity. The oral care formulation of the present invention is chewable to increase gelation speed. Alternatively, mouth rinsing or gargling for oral hygiene is effected by attaching the tablet on a toothbrush and melting and gelling it while maintaining its original shape.

Characteristics of the oral care formulation of the present invention can be summarized as follows:

Firstly, the oral care agent of the present invention is in the form of a tablet prior to contact with water and undergoes in-situ melting and gelling by the chewing action, or water or saliva in the mouth.

Secondly, the tablet undergoing in-situ melting and gelling is prepared by a process involving preparation of high-porous plastic granules, introduction of a water penetration enhancer, and addition of a binder to improve tablet strength and a gelling agent to impart gel-like texture.

Thirdly, the formulation of the present invention uses a sweetening agent, a flavoring agent and the like to achieve pleasing mouth-feel. In addition, active ingredients including fluoride compounds are used to prevent dental caries, periodontal diseases, and dental plaque and calculus deposition by intraoral drug delivery.

### DESCRIPTION OF THE DRAWINGS

The above and other objects, features and other advantages of the present invention will be more clearly understood from the following detailed description taken in conjunction with the accompanying drawings, in which:

FIG. 1 is a schematic view showing a conventional process for producing an in-situ melting and gelling tablet of the present invention;

FIG. 2 is a schematic view showing co-introduction of a gelling agent with a binder solution, upon producing an in-situ melting and gelling tablet of the present invention;

FIG. 3 is a schematic view showing introduction of a gelling agent after preparation of porous plastic granules, upon producing an in-situ melting and gelling tablet of the present invention; and

FIG. 4 is a schematic view showing compression of granules into a tablet after preparation of two types of granules, upon producing an in-situ melting and gelling tablet of the present invention.

### BEST MODE

### Examples

Now, the present invention will be described in more detail with reference to the following examples. These examples are provided only for illustrating the present invention and should not be construed as limiting the scope and spirit of the present invention.

### Examples and Comparative Examples

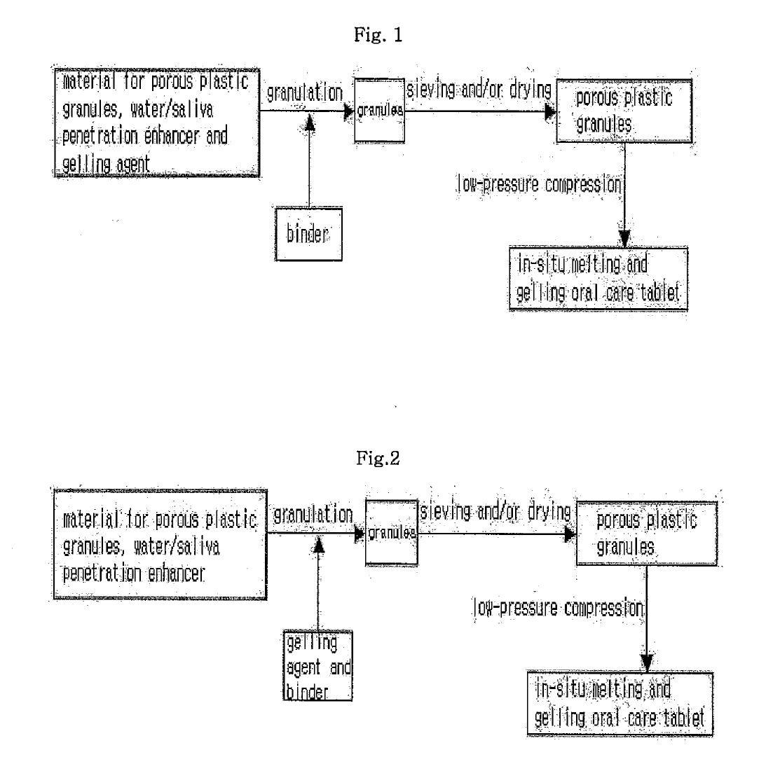

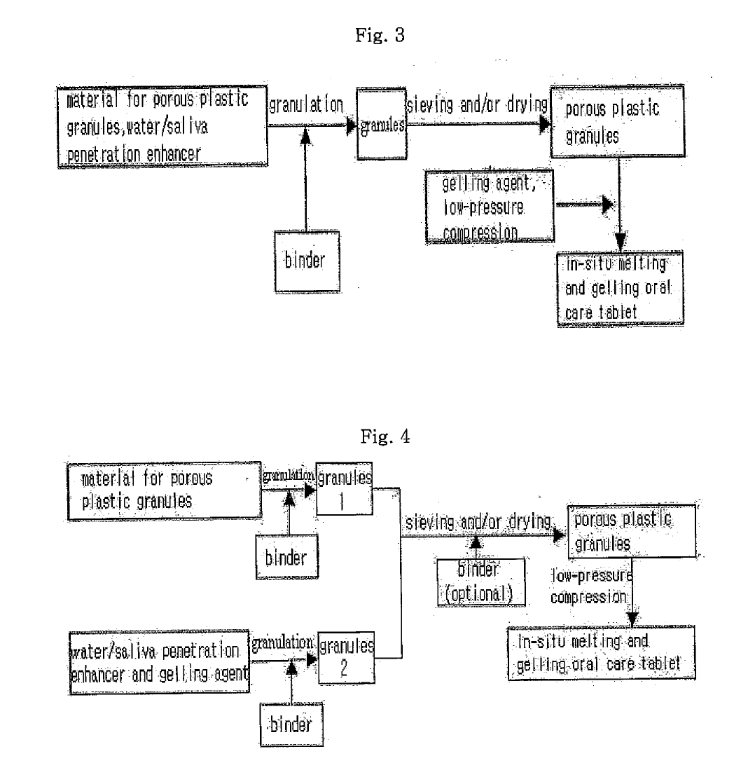

In-situ melting and gelling tablet-type oral care agents of the present invention were prepared according to a manufacturing process shown in FIG. 1.

Table 1 below shows composition ratios of raw materials used for preparation of porous plastic granules in the first aspect of the tablet-type oral care composition according to the present invention. Comparative Examples 1-1 and 1-2 included no gelling agent.

TABLE 1

| | | | | (% by weight) | |
|---|---|---|---|---|---|
| | Examples | | | Comp. Examples | |
| Components | 1-1 | 1-2 to 1-4 | 1-5 | 1-1 | 1-2 |
| Sorbitol | 49.63 | — | 24.81 | 54.53 | — |
| Erythritol | — | 49.63 | 24.82 | — | 54.53 |
| Sodium lauryl sulfate | 3.00 | 3.00 | 3.00 | 3.00 | 3.00 |
| Carbopol | 1.50 | 1.50 | 1.50 | — | — |
| Precipitated silica | 20.00 | 20.00 | 20.00 | 20.00 | 20.00 |
| Calcium carbonate | 20.00 | 20.00 | 20.00 | 20.00 | 20.00 |
| Sodium hydrogen | 1.00 | 1.00 | 1.00 | — | — |
| carbonate | | | | | |
| Tribasic sodium | 0.40 | 0.40 | 0.40 | — | — |
| phosphate | | | | | |
| Xanthan gum | 1.00 | — | 1.00 | — | — |
| Sodium carboxymethyl | — | 1.0 | — | — | — |
| cellulose | | | | | |
| Cross-linked polyvinyl | 3.00 | 3.00 | — | 3.00 | 3.00 |
| pyrrolidone | | | | | |
| Cross-linked cellulose | — | — | 3.00 | — | — |
| Sodium fluoride | 0.22 | 0.22 | 0.22 | 0.22 | 0.22 |
| Saccharin sodium | 0.25 | 0.25 | 0.25 | 0.25 | 0.25 |
| Total | 100.00 | 100.00 | 100.00 | 100.00 | 100.00 |

Table 2 below shows composition ratios of raw materials used for preparation of porous plastic granules in the second aspect of the tablet-type oral care composition according to the present invention. Comparative Example 2-1 included no gelling agent and anti-adhesive agent, whereas Comparative Example 2-2 included no anti-adhesive agent to teeth.

TABLE 2

| | | | | (% by weight) | |
|---|---|---|---|---|---|
| | Examples | | | Comp. Examples | |
| Components | 2-1 | 2-2 to 2-4 | 2-5 | 2-1 | 2-2 |
| Sorbital | 77.13 | — | 28.61 | 52.53 | — |
| Xylitol | — | 72.13 | 28.52 | — | 76.03 |
| Sodium lauryl sulfate | 2.00 | 2.00 | 2.00 | 2.00 | 2.00 |
| Carbopol | 1.50 | 1.50 | 1.50 | — | 1.50 |
| Precipitated silica | 15.00 | 20.00 | — | — | 20.00 |
| Calcium carbonate | — | — | 35.00 | 35.00 | — |
| Sodium hydrogen | 1.00 | 1.00 | 1.00 | — | — |
| carbonate | | | | | |
| Tribasic sodium | 0.40 | 0.40 | 0.40 | — | — |
| phosphate | | | | | |
| Xanthan gum | 1.00 | — | 1.00 | — | — |
| Sodium carboxymethyl | — | 1.0 | — | — | — |
| cellulose | | | | | |
| Cross-linked polyvinyl | 1.00 | — | — | — | — |
| pyrrolidone | | | | | |
| Cross-linked cellulose | — | 1.00 | — | — | — |
| Microcrystalline | — | — | 1.00 | 10.00 | — |
| cellulose | | | | | |
| Sodium fluoride | 0.22 | 0.22 | 0.22 | 0.22 | 0.22 |
| Saccharin sodium | 0.25 | 0.25 | 0.25 | 0.25 | 0.25 |
| Glyceryl monostearate | 0.5 | 0.5 | 0.5 | — | — |
| Total | 100.00 | 100.00 | 100.00 | 100.00 | 100.00 |

Table 3 below shows composition ratios of raw materials used for preparation of porous plastic granules in the third aspect of the tablet-type oral care composition according to the present invention. Comparative Example 3-1 included no gelling agent and anti-adhesive agent, whereas Comparative Example 3-2 included no anti-adhesive agent to teeth and humectant.

TABLE 3

| | | | | (% by weight) | |
|---|---|---|---|---|---|
| | Examples | | | Comp. Examples | |
| Components | 3-1 | 3-2 to 3-4 | 3-5 | 3-1 | 3-2 |
| Sorbitol | 72.13 | — | 26.11 | 52.53 | — |
| Xylitol | — | 67.13 | 26.02 | — | 76.03 |
| Sodium lauryl sulfate | 2.00 | 2.00 | 2.00 | 2.00 | 2.00 |
| Carbopol | 1.50 | 1.50 | 1.50 | — | 1.50 |
| Precipitated silica | 15.00 | 20.00 | — | — | 20.00 |
| Calcium carbonate | — | — | 35.00 | 35.00 | — |
| Sodium hydrogen | 1.00 | 1.00 | 1.00 | — | — |
| carbonate | | | | | |
| Tribasic sodium | 0.40 | 0.40 | 0.40 | — | — |
| phosphate | | | | | |
| Xanthan gum | 1.00 | — | 1.00 | — | — |
| Sodium carboxymethyl | — | 1.00 | — | — | — |
| cellulose | | | | | |
| Cross-linked polyvinyl | 1.00 | — | — | — | — |
| pyrrolidone | | | | | |
| Cross-linked cellulose | — | 1.00 | — | — | — |
| Microcrystalline | — | — | 1.00 | 10.00 | — |
| cellulose | | | | | |
| Glycerin | 5.00 | 5.00 | 5.00 | — | — |
| Sodium fluoride | 0.22 | 0.22 | 0.22 | 0.22 | 0.22 |
| Saccharin sodium | 0.25 | 0.25 | 0.25 | 0.25 | 0.25 |
| Glyceryl monostearate | 0.5 | 0.5 | 0.5 | — | — |
| Total | 100.00 | 100.00 | 100.00 | 100.00 | 100.00 |

Four binder solutions were prepared as shown in Table 4 below.

TABLE 4

| No. | Binder solutions | Amounts |
|---|---|---|
| 1 | 2% aqueous hydroxypropyl cellulose solution | 25 g |
| 2 | 70% aqueous sorbitol solution | 25 g |
| 3 | 2% aqueous hydroxypropylmethyl cellulose solution | 25 g |
| 4 | 2% aqueous carboxymethyl cellulose solution | 25 g |

According to the instruction set forth in Table 5 below, the material for porous plastic granules shown in Tables 1 to 3 were respectively mixed with the binder solutions given in Table 4 to thereby prepare porous plastic granules of Examples 1-1 to 1-5, 2-1 to 2-5 and 3-1 to 3-5, and the porous plastic granules of Comparative Examples 1-1 and 1-2, 2-1 and 2-2, and 3-1 and 3-2.

The thus-obtained porous plastic granules were dried at room temperature for more than 2 hours and compressed into tablets using a tableting machine. For smooth compression, a glidant was added in a ratio of 1 to 2%. Kinds and amounts of the glidants are given in Table 5 below.

TABLE 5

| | Binder | |
|---|---|---|
| Example No. | solutions | Glidants |
| Examples 1-1, 2-1 and 3-1 | 2 | Magnesium stearate 1% |
| Examples 1-2, 2-2 and 3-2 | 1 | Magnesium stearate 2% |
| Examples 1-3, 2-3 and 3-3 | 2 | Magnesium stearate 1% |
| Examples 1-4, 2-4 and 3-4 | 4 | Magnesium stearate 2% |
| Examples 1-5, 2-5 and 3-5 | 3 | Magnesium stearate 1% |
| Comp. Examples 1-1, 2-1 and 3-1 | 1 | Magnesium stearate 1% |
| Comp. Examples 1-2, 2-2 and 3-2 | 2 | Magnesium stearate 2% |

Melting/gelling time was measured for tablets of Examples and Comparative Examples obtained by a compression process: The tablets were placed in a 100 mL beaker filled with 20 mL of water and taken out after 5 sec, 10 sec, 15 sec, 20 sec, 30 sec and 1 min. The tablets were pressed with a spatula and the degree of water penetration into a core of the tablet was observed by naked eyes. In addition, after chewing the tablets 2, 4, 6, 10 and 15 times, mouth-feel for gel texture and chewing feeling were scored on a scale of 0-5, where 5: excellent, 4: good, 3: moderate, 2: poor, and 1: very poor).

Friability of the tablet was evaluated using Electrolab Friabilator (USP: United States Pharmacopeia).

Melting/gelling time and friability of the tablets in the first aspect of tablet-type oral care compositions according to the present invention are given in Table 6 below.

TABLE 6

| Example No. | Melting/gelling time (sec) | Friability |
|---|---|---|
| Example 1-1 | 15 | 0.5% |
| Example 1-2 | 5 | 0.5% |
| Example 1-3 | 5 | 1.0% |
| Example 1-4 | 5 | 1.0% |
| Example 1-5 | 5 | 0.5% |
| Comp. Example 1-1 | not determinable | 0.5% |
| Comp. Example 1-2 | not determinable | 0.5% |

As can be seen from Table 6, Examples 1-1 to 1-5 all exhibited good melting/gelling time with a level of friability comparable to that of Comparative Examples 1-1 and 1-2. That is, the tablets prepared in Comparative Examples 1-1 and 1-2 exhibited the non-determinable melting time of more than 30 seconds, while the tablets of the present invention showed simultaneous melting and gelling within 15 seconds.

Melting/gelling time, chewing feel and friability of the tablets in the second aspect of tablet-type oral care compositions according to the present invention are given in Table 7 below.

TABLE 7

| | Melting/gelling | | |
|---|---|---|---|
| Example No. | time (sec) | Chewing feel | Friability |
| Example 2-1 | 25 | 4 | 0.5% |
| Example 2-2 | 30 | 4 | 0.5% |
| Example 2-3 | 30 | 4 | 1.0% |
| Example 2-4 | 25 | 4 | 1.0% |
| Example 2-5 | 15 | 5 | 0.5% |
| Comp. Example 2-1 | not determinable | 1 | 0.5% |
| Comp. Example 2-2 | not determinable | 2 | 0.5% |

As can be seen from Table 7, Examples 2-1 to 2-5 all exhibited good melting/gelling time with a level of friability comparable to that of Comparative Examples 2-1 and 2-2. That is, the tablets of Comparative Examples 2-1 and 2-2 exhibited the non-determinable melting time of more than 30 seconds, while the tablets of the present invention showed concurrent melting and gelling within 30 seconds.

Regarding evaluation of mouth-feel before and after chewing the tablets, the formulations of Examples 2-1 to 2-5 all acquired good scores, while the formulations of Comparative Examples 2-1 and 2-2 without addition of the anti-adhesive agent exhibited poor or very poor mouth-feel.

Melting/gelling time, chewing feel and friability of the tablets in the third aspect of tablet-type oral care compositions according to the present invention are given in Table 8 below.

TABLE 8

| | Melting/gelling | | |
|---|---|---|---|
| Example No. | time (sec) | Chewing feel | Friability |
| Example 3-1 | 25 | 4 | 0.5% |
| Example 3-2 | 30 | 4 | 0.5% |
| Example 3-3 | 30 | 4 | 1.0% |
| Example 3-4 | 25 | 4 | 1.0% |
| Example 3-5 | 15 | 5 | 0.5% |
| Comp. Example 3-1 | not determinable | 1 | 0.5% |
| Comp. Example 3-2 | not determinable | 2 | 0.5% |

As can be seen from Table 8, Examples 3-1 to 3-5 all exhibited good melting/gelling time with a level of friability comparable to that of Comparative Examples 3-1 and 3-2. That is, the tablets of Comparative Examples 3-1 and 3-2 exhibited the non-determinable melting time of more than 30 seconds, while the tablets of the present invention showed concurrent melting and gelling within 30 seconds.

Regarding evaluation of mouth-feel before and after chewing the tablets, the formulations of Examples 3-1 to 3-5 all acquired good scores, while the formulations of Comparative Examples 3-1 and 3-2 without addition of the anti-adhesive agent exhibited poor or very poor mouth-feel. Particularly, the formulation of Comparative Example 3-2 exhibited a very slow melting time with the subject tablet being caught between teeth.

Next, the temporal stability of tablets prepared in Examples and Comparative Examples was examined by naked eyes after temperature aging of the tablets and evaluated based on the following criteria. The results thus obtained are given in Tables 9 to 11:

⊚ stable (superficial discoloration and morphological changes not observed)

∘: relatively stable (superficially slight discoloration observed without causing quality problems)

Δ relatively unstable (superficial discoloration and morphological changes observed)

x: very unstable

TABLE 9

| | | RT | 40° C. | 50° C. | 60° C. |
|---|---|---|---|---|---|
| Example 1-5 | 2 weeks | ⊚ | ⊚ | ⊚ | ⊚ |
| | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |
| Comparative | 2 weeks | ⊚ | ⊚ | ⊚ | ⊚ |
| Example 1-1 | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |

TABLE 10

| | | RT | 40° C. | 50° C. | 60° C. |
|---|---|---|---|---|---|
| Example 2-5 | 2 weeks | ⊚ | ⊚ | ⊚ | ⊚ |
| | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |
| Comparative | 2 weeks | ⊚ | ⊚ | ⊚ | ⊚ |
| Example 2-1 | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |

TABLE 11

| | | RT | 40° C. | 50° C. | 60° C. |
|---|---|---|---|---|---|
| Example 3-5 | 2 weeks | ⊚ | ⊚ | ⊚ | ⊚ |
| | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |
| Comparative | 2 weeks | ⊚ | ⊚ | ⊚ | ⊚ |
| Example 3-1 | 4 weeks | ⊚ | ⊚ | ⊚ | ◯ |
| | 6 weeks | ⊚ | ⊚ | ⊚ | ◯ |
| | 8 weeks | ⊚ | ⊚ | ◯ | ◯ |

As can be seen from Tables 9 to 11, the temporal stability of compositions prepared in Examples of the present invention was comparable to that of Comparative Examples.

### INDUSTRIAL APPLICABILITY

As apparent from the above description, the in-situ melting and gelling oral care agent of the present invention is characterized by a tablet formulation unlike conventional ointment-like or liquid products. The tablet-type oral care agent of the present invention is used for oral health maintenance. That is, the oral care agent may be employed for gargling or mouth washing via melting and gelling by water or saliva, or by chewing action following direct administration of the agent to the mouth. Like conventional oral care products, the oral care agent of the present invention may be formulated into a gel-like oral care agent by placing the product on a toothbrush and flowing water thereto. As such, the formulation of the present invention advantageously reduces inconvenience of conventional liquid or ointment-like oral care products and also provides easy portability.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A tablet-type oral care composition which is prepared by compressing porous plastic granules consisting essentially of a material for porous plastic granules, a binder, a gelling agent and a water penetration enhancer under a pressure of 500 kg/cm² or less and undergoes in-situ melting and gelling by water or saliva in oral cavity.

2. A tablet-type oral care composition which is prepared by compressing porous plastic granules consisting essentially of a material for porous plastic granules, a binder, a gelling agent, a water penetration enhancer and an anti-adhesive agent to teeth under a pressure of 500 kg/cm² or less and undergoes in-situ melting and gelling by chewing in the presence of water or saliva in oral cavity.

3. A tablet-type oral care composition which is prepared by compressing porous plastic granules consisting essentially of a material for porous plastic granules, a binder, a gelling agent, a water penetration enhancer, an anti-adhesive agent to teeth and a humectant under a pressure of 500 kg/cm² or less and undergoes in-situ melting and gelling by water or saliva in oral cavity, or by chewing.

4. The oral care composition according to any one of claims 1 to 3, wherein the composition undergoes gelling within 30 seconds.

5. The oral care composition according to any one of claims 1 to 3, wherein a content of the material for porous plastic granules is in the range of 20 to 95% by weight, based on the total weight of the composition.

6. The oral care composition according to any one of claims 1 to 3, wherein a content of the binder is in the range of 1 to 90% by weight based on the total weight of the composition.

7. The oral care composition according to any one of claims 1 to 3, wherein a content of the gelling agent is in the range of 0.1 to 10% by weight, based on the total weight of the composition.

8. The oral care composition according to any one of claims 1 to 3, wherein a content of the water penetration enhancer is in the range of 20 to 80% by weight, based on the total weight of the composition.

9. The oral care composition according to claim 2 or 3, wherein a content of the anti-adhesive agent to teeth is in the range of 0.01 to 10% by weight, based on the total weight of the composition.

10. The oral care composition according to claim 3, wherein a content of the humectant is in the range of 0.01 to 20% by weight, based on the total weight of the composition.

11. The oral care composition according to any one of claims 1 to 3, wherein the material for porous plastic granules is selected from the group consisting of fructose, lactitol, maltitol, maltose, mannitol, sorbitol, sucrose, erythritol, xylitol, maltodextrin, dextrin, ethyl cellulose, polymethylmethacrylate, pregelatinized starch, gum arabic, xanthan gum and its derivatives, guar gum and its derivatives, seaweed gum, carrageenan, dextran, gelatin, alginate, pectin, starch and starch derivatives, cellulose ester, homo- or co-polymers of an unsaturated acid, homo- or co-polymers of an unsaturated amide, homo- or co-polymers of acrylic imine, a vinyl polymer, homo- or co-polymers of a vinyl ester, alkylglycol, polyalkylene oxide, oxyethylene alkyl ester, dextrate, dextrin, dextrose, microcrystalline cellulose, silicified microcrystalline cellulose, powdered cellulose, cellulose acetate, calcium sulfate, calcium carbonate, dibasic calcium phosphate, tribasic calcium phosphate, a calcium salt of carboxymethyl cellulose, silica and mixtures thereof.

12. The oral care composition according to any one of claims 1 to 3, wherein the water penetration enhancer is selected from the group consisting of dextrate, dextrin, dextrose, fructose, lactitol, lactose, maltitol, mannitol, sorbitol, sucrose, erythritol, xylitol, microcrystalline cellulose, silicified microcrystalline cellulose, powdered cellulose, cellulose acetate, calcium sulfate, calcium carbonate, silica, dibasic calcium phosphate, tribasic calcium phosphate, a calcium salt of carboxymethyl cellulose, cross-linked polyvinyl pyrrolidone and mixtures thereof.

13. The oral care composition according to any one of claims 1 to 3, wherein the binder is selected from the group consisting of dextrate, dextrin, dextrose, fructose, lactitol, lactose, maltitol, mannitol, sorbitol, sucrose, erythritol, xylitol, acacia, alginic acid, carbomer (Carbopol), carboxymethyl cellulose, cellulose, dextrin, ethyl cellulose, gelatin, hydroxyethyl cellulose, hydroxypropyl cellulose, hydroxypropylmethyl cellulose, methyl cellulose, polydextrose, polyethylene oxide, polyvinyl pyrrolidone, sodium alginate and mixtures thereof.

14. The oral care composition according to any one of claims 1 to 3, wherein the gelling agent is selected from the group consisting of acacia gum, agar gum, gellan gum, guar gum, pectin, gelatin, alginic acid, sodium alginate, carboxymethyl cellulose, a polyvinyl maleic acid/maleic anhydride copolymer, Carbopol, polyethylene glycol, polyvinyl pyrrolidone, polyethylene oxide, xanthan gum, carrageenan, super porous hydrogel and mixtures thereof.

15. The oral care composition according to claim 2 or 3, wherein the anti-adhesive agent to teeth is glyceryl monooleate, glyceryl monostearate or a mixture thereof.

16. The oral care composition according to claim 3, wherein the humectant is glycerin, polyethylene glycol, propylene glycol, sorbitol or mixtures thereof.

17. The oral care composition according to any one of claims 1 to 3, further comprising 0.001 to 10% by weight of at least one material selected from an anti-caries agent, an anti-calculus agent, an agent for prevention of periodontal diseases, an antibacterial agent, an anti-inflammatory agent and a teeth whitening agent.

18. The oral care composition according to any one of claims 1 to 3, further comprising 0.001 to 10% by weight of a forming agent.

19. The oral care composition according to any one of claims 1 to 3, further comprising 5 to 70% by weight of an abrasive.

20. The oral care composition according to any one of claims 1 to 3, further comprising at least one of 0.001 to 10% by weight of a flavoring agent and 0.001 to 20% by weight of a sweetening agent, based on the total weight of the composition.

21. The oral care composition according to any one of claims 1 to 3, further comprising 0.1 to 5% by weight of a lubricant.
